(19) United States
(12) Patent Application Publication
(10) Pub. No.: US 2007/0191787 A1
(43) Pub. Date: Aug. 16, 2007

(54) Medical treatment control system

(75) Inventors: Bernard C.B. Lim, Oakville (CA); Taras Worona, Etobicoke (CA); Davis A.R. Kanbergs, Milton (CA); Mark P. Costa, Milton (CA); Hao Chen, Mississauga (CA); Roy T. Zhao, Brampton (CA); Gabriele Klein, Mississauga (CA); Kathleen Chancellor-Maddison, Mississauga (CA); David G. Matsuura, Encinitas, CA (US); Philip J. Simpson, Escondido, CA (US); Walter D. Gillespie, San Diego, CA (US); John N. Mitchell, Goffs Oak (GB)

Correspondence Address: FOLEY & LARDNER LLP, 1530 PAGE MILL ROAD, PALO ALTO, CA, 94304, US

(73) Assignee: Vasogen Ireland Limited

(21) Appl. No.: 10/536,651

(22) PCT Filed: Nov. 26, 2003
(86) PCT No.: PCT/CA03/01838

§ 371 Date: Jan. 20, 2006

Related U.S. Application Data
(60) Provisional application No. 60/428,942, filed on Nov. 26, 2002.
(60) Provisional application No. 60/464,659, filed on Apr. 23, 2003.
(60) Provisional application No. 60/482,725, filed on Jun. 27, 2003.

(51) Int. Cl. A61M 5/00

(52) U.S. Cl. 604/246

(57) ABSTRACT

A device for controlling the collection and delivery of materials to a patient, the device has a dispenser-engaging portion, the dispenser-engaging portion being operable in a release position to receive a materials dispenser when the dispenser is in a materials-containing configuration, the dispenser-engaging portion being operable in a lock condition for locking the dispenser therewith, and access control means for controlling the release and lock positions according to a material transaction condition.

### REFERENCE TO CO-PENDING APPLICATIONS

The entire subject matter of U.S. Provisional application Ser. No. 60/428,942 filed Nov. 26, 2007 and entitled BLOOD TREATMENT CONTROL SYSTEM is incorporated by reference. The applicant claims priority benefit under Title 35, United States Code, Section 119(e) of U.S. Provisional application Ser. No. 60/429,942 filed Nov. 26, 2002 and entitled BLOOD TREATMENT CONTROL SYSTEM.

The entire subject matter of U.S. Provisional application Ser. No. 60/464,659 filed Apr. 23, 2003 and entitled DISPENSING SYSTEMS is incorporated by reference. The applicant claims priority benefit under Title 35, United States Code, Section 119(e) of U.S. Provisional application Ser. No. 60/464,659 filed Apr. 23, 2003 and entitled DISPENSING SYSTEMS.

The entire subject matter of U.S. Provisional application Ser. No. 60/482,725 filed Jun. 27, 2003 and entitled MEDICAL TREATMENT CONTROL SYSTEM is incorporated by reference. The applicant claims priority benefit under Title 35, United States Code, Section 119(e) of U.S. Provisional application Ser. No. 60/482,725 filed Jun. 27, 2003 and entitled MEDICAL TREATMENT CONTROL SYSTEM.

### BACKGROUND OF THE INVENTION

1. Field of the Invention

The present invention relates to the controlling of medical treatments.

2. Description of the Related Art

The field of medicine has worked diligently over the years to improve the safety of blood collection and delivery in patient care. The consequences of an error, for example in delivering a blood sample to the wrong patient, can be serious, if not severe. A current technique uses a series of four identically numbered decals that are used to track the two syringes, the disposable and the patient during the blood treatment. The blood treatment involves:
1 Removing the blood from the patient to a syringe.
2 Transporting that syringe to a blood treatment disposable within which the treatment occurs.
3 Removing the treated blood from the disposable to the blood delivery syringe and
4 Returning the blood to the patient

The serial number on the decals are compared to one another at each transfer step by the operator to ensure that the correct blood is tracked throughout the process to eventually ensure the correct blood is given to each patient.

At each comparison a decision is made and at each decision point there exists a potential for human error.

An intensive Care Unit (ICU) at a British hospital has gained recognition for its efforts to reduce errors in blood treatments. The system requires that a new patient entering a ward on the ICU receive a new wristband that contains the information of Date of Birth (DOB), Name, Hospital ID number and a 2D barcode that contains the same information as well as any allergies, blood type and medications that the patient is currently receiving. This information is also stored on the hospital database.

The ICU nurse can then order autologous or donated blood to be delivered to the ICU for the patient. The blood information is confirmed on the blood bank computer monitor and the correct blood is selected. New barcodes are printed and placed on those blood bags. The blood bags are delivered to the patient.

At the bedside, the blood bag barcode and patient barcode are scanned to see if the blood and patient match If they match, the operator or nurse is granted approval to proceed with the transfusion. If the match is not made, the nurse is not provided with approval and is given a warning not to transfuse the blood. The barcode reader and printed labels facilitates a machine assisted blood matching.

Despite the advances that have been made in the control of medical treatments, improvements are still needed.

### SUMMARY OF THE INVENTION

In one of its aspects, the present invention provides a device for controlling the collection and delivery of blood, comprising a syringe-engaging portion, the syringe-engaging portion being operable in a release position to receive a syringe when the syringe is in a blood-containing configuration, the syringe-engaging portion being operable in a lock position for locking the syringe therewith, and access control means for controlling the release and lock positions according to a blood transaction condition.

In one embodiment, the syringe-engaging portion has a side wall containing a cavity to receive the syringe. The syringe is of the type having a body having a first end flange on one end thereof and a plunger slidably engaged with the body, the plunger having a second end flange on a remote end thereof the cavity having a first formation to receive the first end flange.

In one embodiment, the access control means further comprises at least one barrier portion to extend at least partially across the cavity in the lock position. In one example, the access control means has a pair of barrier members with opposing free end regions, the barrier members being movable between an open position wherein the free ends are separated to permit the syringe to pass therebetween and a closed position wherein the free ends are positioned sufficiently close to one another to prevent the removal or the addition of the syringe from the cavity. The barrier members are pivotally coupled to the syringe-engaging portion.

In one embodiment, the device has a control portion, the syringe-engaging portion being removably attached to the control portion. Actuating means are mounted in the control portion and are releasably coupled to the barrier members for actuating the barrier members between the open and closed positions. In addition, a second lock means is provided for locking the syringe engaging portion with the control portion, for reasons which will be described herein below.

In one embodiment, the control portion includes a data transfer unit. The data transfer unit is operable to receive patient identification data representative of a subject patient and thereby to establish a first blood transaction condition, the control portion being operable in the first blood transaction condition to transfer the barrier members to the release position to receive a first syringe containing blood from the subject patient and to transfer the barrier members to the lock position to lock the first syringe in the cavity.

In one embodiment, the data transfer unit includes data transmitting means, data receiving means and data storage means for recording data received by the data receiving means. Either the data transmitting means, the data receiving means, or both, may each include a wired or wireless data port. The wireless data port may include, for example, a barcode reader, or an RF signal receiver.

In one embodiment, the data transfer unit is operable to transfer the patient identification data to a blood treatment unit and thereby to establish a second blood transaction condition, the control portion being operable in the second blood transaction condition in which the barrier members are movable to the release position to release the first syringe to a first syringe station in the blood treatment unit.

In one embodiment, the data transfer unit is operable to receive treated blood identification data from the blood treatment unit, the data transfer unit also being operable to receive treated blood verification data from a second syringe containing treated blood from the subject patient and positioned at a second syringe station in the blood treatment unit, thereby to establish a third blood transaction condition, the control portion being operable in the third blood transaction condition to transfer the barrier members to the release position to receive the second syringe.

In this case, the second lock means is operable to release the syringe engaging portion at the end of a blood treatment procedure to permit the second syringe to be transported to the subject patient while still being positioned in the syringe engaging portion.

In one embodiment, the data transfer unit is operable to receive patient verification data to establish a fourth blood transaction condition, the control portion being operable in the fourth blood transaction condition wherein the barrier members are movable to the release position to release the second syringe.

In another of its aspects, there is provided a system for blood processing, comprising:
a first syringe to receive a blood sample from a subject patient;
a patient identifier attachable to the subject patient;
a blood treatment unit;
a syringe carrier for transferring the first syringe containing the blood sample to the blood treatment unit, the syringe carrier being operable in a release position to receive the first syringe when the first syringe is in a blood-containing configuration, the syringe carrier being operable in a lock position for locking the first syringe therewith, and access control means for controlling the release and lock positions to control access to the first syringe according to a blood sample transfer condition.
a second syringe to receive the blood sample after treatment in the blood treatment unit to form a treated blood sample; and
the syringe carrier being operable in the release position to receive the second syringe when the second syringe is in a blood-containing configuration, the syringe carrier being operable in the lock position for locking the second syringe therewith, said access control means being operable to controlling the release and lock positions to control access to the second syringe according to a treated blood transfer condition.

Preferably, the syringe carrier has provision to receive or record indicia indicative of a patients name or other patient identifying data, in manner readable to the operator or the patient or both. Preferably, the syringe carrier is also provided with a mechanism allowing the operator to transfer the carrier to the release position to release the syringe.

In still another of its aspects, there is provided a method of controlling the transfer of blood between a subject patient and a blood treatment unit, comprising the steps of:
providing a first syringe containing a sample of untreated blood from a subject patient;
providing a syringe carrier which is operable in a release position to receive the first syringe; the syringe carrier being operable in a lock position for locking the first syringe therewith, the carrier having an access controller for controlling the release and lock positions according to a blood transaction condition, the access controller including a data transfer unit which is operable to receive patient identification data representative of a subject patient;
in a first blood transaction step, delivering patient identification data representative of a subject patient to the data transfer unit, thereby to place the syringe carrier in a release position to receive the first syringe and thereafter to place the syringe carrier in a lock position to lock the first syringe therein;
in a second blood transaction step, transferring the patient identification data to a blood treatment unit, thereby to place the syringe carrier in the release position to release the first syringe to a first syringe station in the blood treatment unit;
in a third blood transaction step, delivering treated blood identification data from the blood treatment unit to the syringe carrier, and delivering treated blood verification data from a second syringe containing treated blood from the subject patient and which is positioned at a second syringe station in the blood treatment unit, and placing the syringe carrier in the release position to receive the second syringe; and
in a fourth blood transaction step, delivering patient verification data to the syringe carrier and placing the syringe carrier in the release position to release the second syringe.

In still another of its aspects, the present invention provides a process of extracting a body fluid aliquot from a patient, extracorporeally treating at least a portion of the aliquot and returning the treated portion to said patient, comprising the steps of:
equipping the patient with a body fluid aliquot identification means which includes patient-identifying indicia;
withdrawing the body fluid aliquot from the patient;
labeling the aliquot or portion thereof to be treated with aliquot-identifying indicia uniquely correlating with said patient-identifying indicia;
extracorporeally treating the labeled aliquot or portion thereof;
establishing correlation between the aliquot-identifying indicia and said patient-identifying indicia in order to permit patient access to the treated aliquot or portion thereof; and
after establishing said correlation, returning the treated aliquot or portion thereof to the patient;
whereby said patient is assured of receiving a treated aliquot or portion thereof which was initially extracted from said patient.

Preferably, the aliquot is whole blood and the entire aliquot as withdrawn is treated (i.e. no fractionation step).

Preferably, the aliquot is withdrawn into a first dispenser carrying the aliquot-identifying indicia and then is transferred from said first dispenser into a treatment container for conducting the treatment. In this case, the treatment container is labeled to provide a first treated-aliquot-identifying indicia for the treated aliquot. Following treatment, the aliquot is transferred from the treatment container to a second dispenser after treatment. Similarly, the second dispenser is labeled to provide a second treated-aliquot-identifying indicia for the treated aliquot. The second treated-aliquot-identifying indicia is checked to correlate with the patient-identifying indicia to provide patient access to the treated aliquot for return to the patient.

Preferably, the blood aliquot is treated with oxidative stress which, in one instance, is ozone/oxygen gaseous mixture which is bubbled through the aliquot. In another case, the blood aliquot is treated with UV radiation, or heat, or at least two of UV, oxygen/ozone and heat. In another example, the blood aliquot is treated with UV, oxygen/ozone and heat.

Preferably, the first and/or second dispensers include syringes or syringe-type devices, but may be applied to a range of other dispensers as well.

In still another of its aspects, the present invention provides a device for controlling the collection and delivery of materials to a patient, comprising a dispenser-engaging portion, the dispenser-engaging portion being operable in a release position to receive a materials dispenser when the dispenser is in a materials-containing configuration, the dispenser-engaging portion being operable in a lock position for locking the dispenser therewith, and access control means for controlling the release and lock positions according to a material transaction condition.

Preferably, the dispenser includes a syringe, IV bottle, powder and/or atomized fluid and/or gas inhalant dispenser, implant delivery dispenser, ventilator, syringe pump, intubation tube, or a gastrointestinal feeding tube or a plurality and/or a combination thereof.

Preferably, the dispenser-engaging portion includes a first cavity to receive the dispenser, the first cavity being accessible through a side wall or an end wall thereof. In one example, the dispenser-engaging portion has a side wall and the first cavity is located in the side wall. In this case, the access control means further comprises at least one barrier portion to extend at least partially across the first cavity in the lock position.

In still another of its aspects, the present invention provides a process of extracting a body fluid aliquot from a patient, extracorporeally treating at least a portion of the aliquot and returning the treated portion to said patient, comprising the steps of:
equipping the patient with a body fluid aliquot identification means which includes patient-identifying indicia;
withdrawing the body fluid aliquot from the patient;
labeling the aliquot or portion thereof to be treated with aliquot-identifying indicia uniquely correlating with said patient-identifying indicia;
locking the aliquot against delivery with an indicia responsive lock;
extracorporeally treating the labeled aliquot or portion thereof;
establishing correlation between the aliquot-identifying indicia and said patient-identifying indicia in order to permit patient access to the treated aliquot or portion thereof; and
after establishing said correlation, returning the treated aliquot or portion thereof to the patient by response of the indicia responsive lock to the correlation so established;
whereby said patient is assured of receiving a treated aliquot or portion thereof which was initially extracted from said patient.

In a further aspect, the present invention provides a device for controlling the delivery of blood, comprising a syringe-engaging portion, the syringe-engaging portion being operable in a lock position for locking the syringe therewith when the syringe is in a blood-containing configuration, and in a release position to release the syringe, and access control means for controlling the release and lock positions according to a blood transaction condition.

In one embodiment, the syringe-engaging portion includes a cavity to receive the syringe.

Preferably, the access control means further comprises at least one barrier portion to extend at least partially across the cavity in the lock position.

In one embodiment, the access control means includes a data transfer unit, the data transfer unit being operable to receive patient identification data representative of a subject patient and thereby to establish a blood transaction condition, the control portion being operable in a blood transaction condition to establish the release position for the barrier member to release the syringe.

In one embodiment, the data transfer unit includes data receiving means and data storage means for recording data received by the data receiving means. The data receiving means may include a wired or wireless data port, wherein the latter may include a barcode reader, an RF signal receiver or an Infrared transmitter receiver. The data transfer unit is operable to receive patient verification data to establish the release position for the barrier member to release the syringe.

In yet a further of its aspects, the present invention provides a method of controlling the transfer of blood between a subject patient and a blood treatment unit, comprising the steps of:
providing a first syringe to receive a sample of untreated blood from a subject patient;
providing the subject patient with a patient RF signal processor;
providing a second syringe to receive the sample following treatment;
providing each of the first syringe and the second syringe with an RF signal processor;
arranging the RF signal processors on the first syringe and with the patient to issue a signal containing common or related identity data;
delivering the first syringe to the blood treatment unit for performing a treatment step to form a treated blood sample;
reading the identity data from the first syringe and writing the identity data to the second syringe;
collecting the treated blood sample from the treatment unit in the second syringe;
bringing the second syringe within range of the patient RF signal processor to confirm a match therebetween; and thereafter
delivering the treated blood sample to the patient.

### BRIEF DESCRIPTION OF THE DRAWINGS

Several preferred embodiments of the present invention will now be described, by way of example only, with reference to the appended drawings in which:

FIG. 1 is a schematic view of a medical treatment involving a medical treatment control system;

FIG. 2 is a perspective view of a several portions of the system of FIG. 1;

FIG. 3 presents several views of a component of the system of FIG. 1;

FIGS. 3a to 3d present several additional views of the component of FIG. 3;

FIG. 4 presents opposite end views of the component of FIG. 3;

FIG. 5 shows several assembly views of the component of FIG. 3;

FIG. 5a to 5c present several additional views of the component of FIG. 3;

FIG. 5d is a schematic view of several operative positions of the component of FIG. 3.

FIG. 6 is a perspective view of two components of the system of FIG. 2;

FIGS. 6a to 6c present several additional views of one component shown in FIG. 6; and

FIGS. 7 to 9 present views of other components of FIG. 1.

### DESCRIPTION OF THE PREFERRED EMBODIMENTS

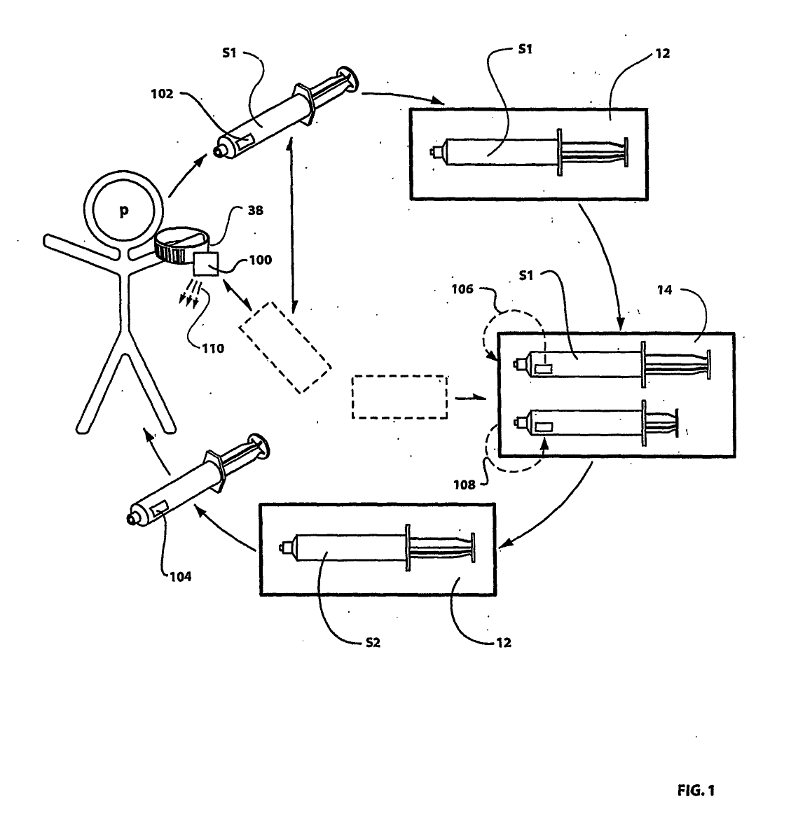

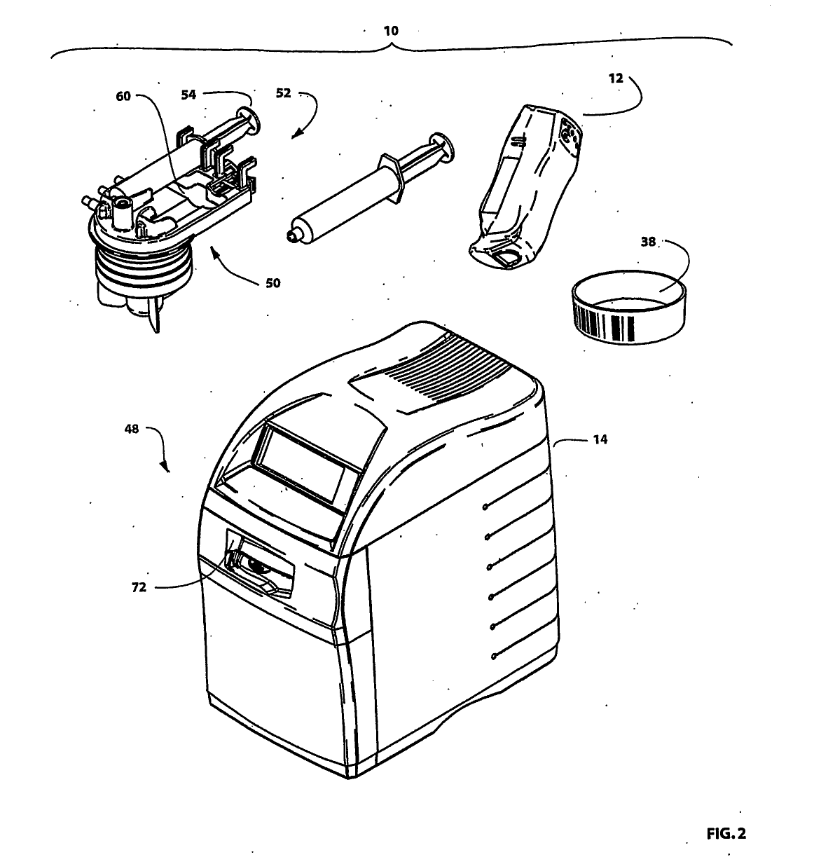

FIG. 1 of the accompanying drawings is a flow chart of a specific preferred embodiment of the process of the present invention applied to the extraction of blood from a patient P, extracorporeal treatment of blood by way of a treatment unit 14 and return of the treated blood to the same patient P. In other words, this is an autologous blood treatment process in which it is vital that the treated blood be returned to the same patient P from whom the blood was extracted. In this process, the patient P is provided with a wristband 38 which includes an identification unique to the patient. A first syringe S1 is used to extract blood from the patient, and is coded for unique interaction with an ID system on the wristband 38. The first syringe S1 containing an aliquot of the patient's blood is then received in a syringe carrier 12 which is capable of receiving data to verify the identity of the patient P and to lock the syringe therein. The syringe carrier 12 is also equipped to deliver the syringe S1 to a treatment unit 14, which withdraws the blood from syringe S1 and performs a designated treatment on the blood. The treatment unit 14 then provides the treated blood in a second syringe S2 which is received by the syringe carrier 12 and returned to the patient for administering the treated blood thereto. In both cases, the syringe carrier 12 receives and/or transmits data to both the wrist band 38 (or its equivalent) and the blood treatment unit 14. Furthermore, the wrist band 38, the syringe carrier 12, the treatment unit 14, and/or an intermediate unit may be used to generate an audit record of the blood treatment. Also included are RF ID chips 100, 102, 104 associated respectively with the wrist band 38, syringe S1 and syringe S2, to be read and matched by outputs 106, 108 and 110 as described below.

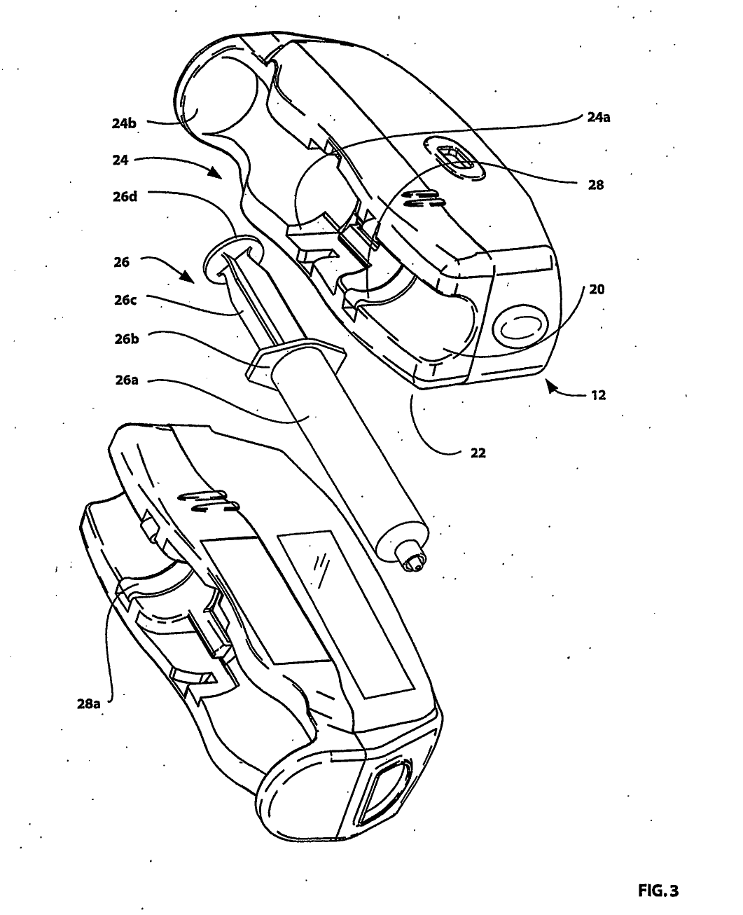

FIG. 2 illustrates some of the components used in the process of FIG. 1 to provide a system 10 for controlling the collection and delivery of materials to a patient. In this particular example, the system is used in connection with blood processing, it being understood that the present system and method may be applied to other medical treatments and dispensers therefor. The system is based on the control of both the collection from and the delivery of blood to, the patient. The system 10 includes a dispenser engaging portion, in the form of a syringe carrier 12 which controls the transfer of a blood sample contained in a syringe, between the patient and a blood treatment unit 14. As will be described, the system is predicated on the concept that the administration of the technique can, in one embodiment, be governed by the syringe carrier 12 which serves as a key to the blood treatment unit 14. In other words, any attempt to avoid using the syringe carrier 12 will render the system 10 inoperable. The syringe carrier 12 is capable of receiving data to verify the identity of a subject patient and/or the blood sample at predetermined stages of a blood treatment and then to lock, within its grasp, a syringe containing blood, either untreated or treated, from or to the subject patient, in a manner which renders the syringe inaccessible and inoperable until proper verification has been made. However, the syringe carrier is not necessary in all cases. One embodiment discussed below provides for a secure method of verifying the patient and the blood during the blood treatment by the use of RF ID tags on a wristband and two syringes, wherein the wristband includes an RF reader and an RF writer.

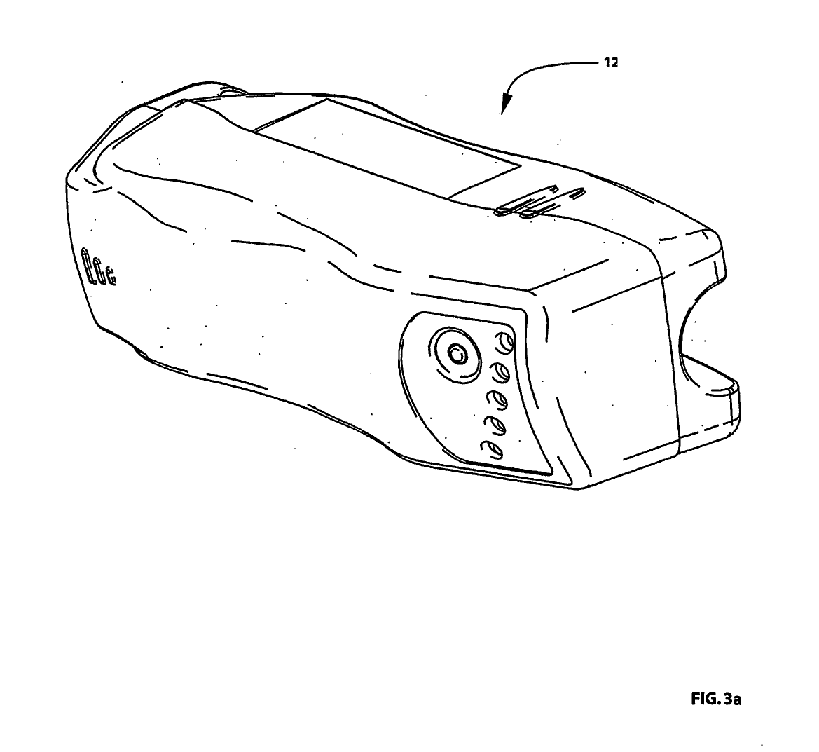

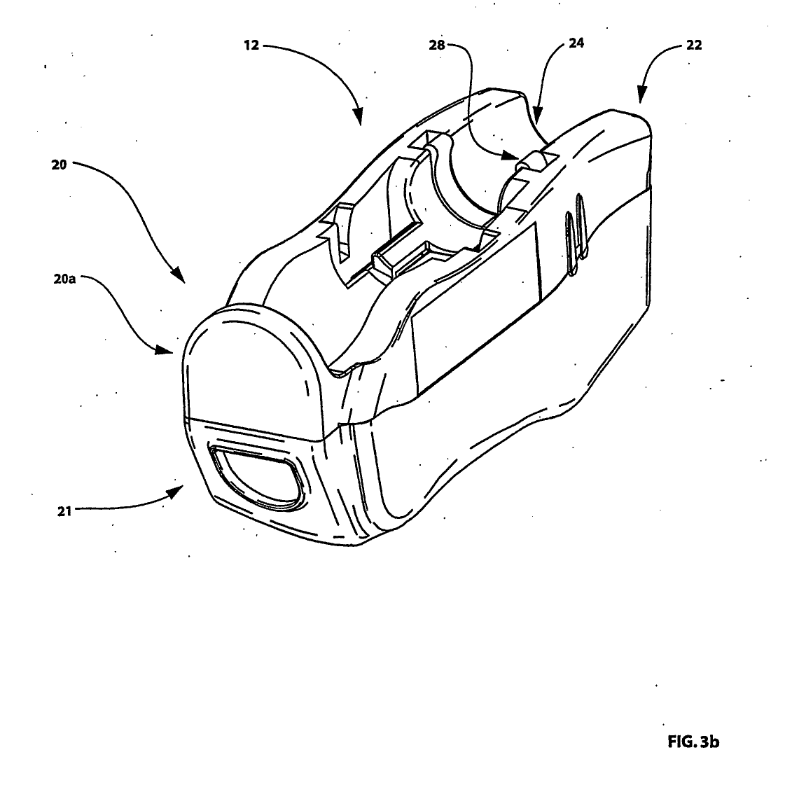

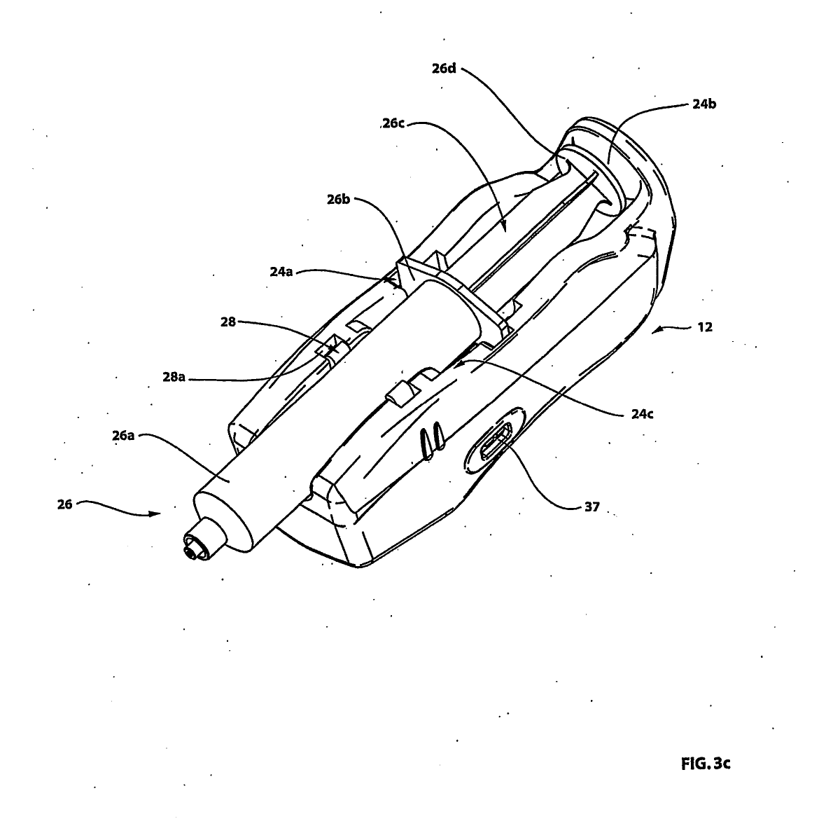

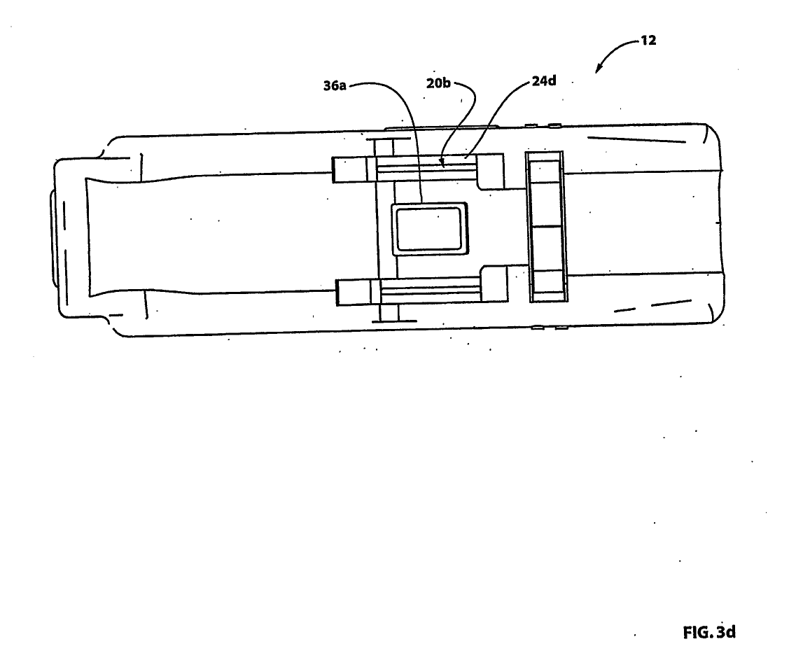

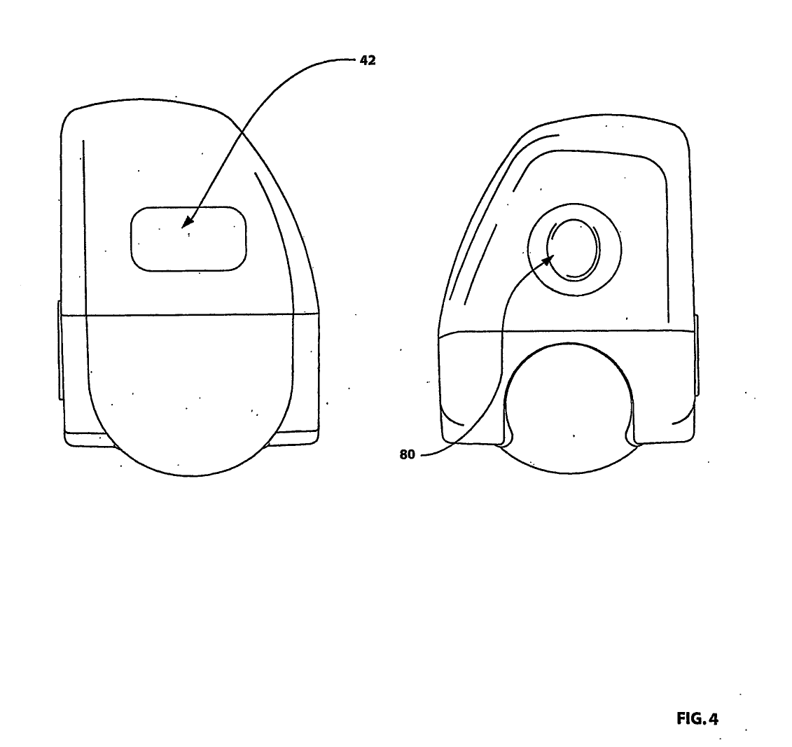

FIGS. 3, and 3a to 3c show more details of the syringe carrier 12. As best seen in FIGS. 3b and 3c, the syringe carrier 12 has a syringe-engaging portion 20 and a control portion 21 interfitting therewith and detachable therefrom. The syringe engaging portion 20 is operable in a release position to receive a sample-containing syringe 26 when the syringe is in a blood-containing configuration. The syringe-engaging portion is operable in a lock position for locking the syringe 26 therewith and is coupled with the control portion 21 as will be described. The syringe carrier 12 has, as will be explained, an access control means for controlling these release and lock positions according to a blood transaction condition.

As best seen in FIGS. 3b and 3c, the syringe-engaging portion 20 has a side wall 22 containing a cavity 24 to receive the syringe 26, the latter having a body 26a with a first end flange 26b on one end thereof and a plunger 26c slidably engaged with the body 26a and having a second end flange 26d on a remote end thereof. In this case, the cavity 24 has a first formation 24a to receive the first end flange 26b and a second formation 24b to receive the second end flange 26d.

The first formation 24a is dimensioned so that it closely approximates the outer profile of the first end flange 26b, while the second formation 24b is in the form of a rear end wall which is shaped to shield the second end flange 26b from being inadvertently contacted by an obstruction. Thus, when the syringe is placed in the cavity, the plunger 26c is less likely to be unintentionally depressed relative to the body 26a, to cause an unwanted dispensing of blood from the syringe, while in the syringe carrier 12. Alternatively, the second formation 24b may be arranged to engage the second flange 26d so that the plunger cannot be moved relative to the body, thereby causing both the plunger and the body to be held in position, thereby preventing the syringe 26 from being removed from the syringe carrier 12 and preventing the blood sample from being dispensed from the first syringe S1 while in the syringe carrier 12.

Again, referring to FIGS. 3b and 3c, the syringe carrier 12 has access control means which includes a pair of barrier members 28 with opposing free ends 28a. The barrier members 28 are movable between an open release position wherein the free ends 28a are sufficiently separated to permit the syringe 26 to pass therebetween and a lock position wherein the free ends 28a extend toward one another at least partially across the cavity and are locked in that position to prevent removal of the syringe 26 from the cavity. In this case, the barrier members 28 are arcuate in shape and are pivotally coupled to the syringe-engaging portion near opposite sides of the cavity 24, but may also be pivotally or otherwise mounted to the syringe engaging portion 20.

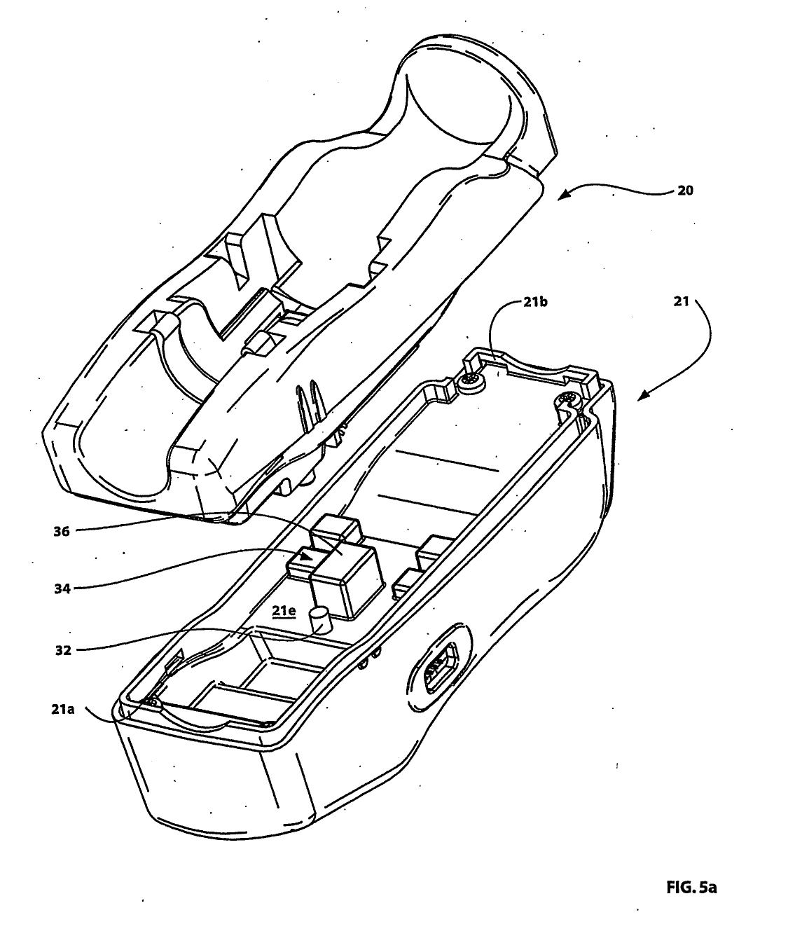

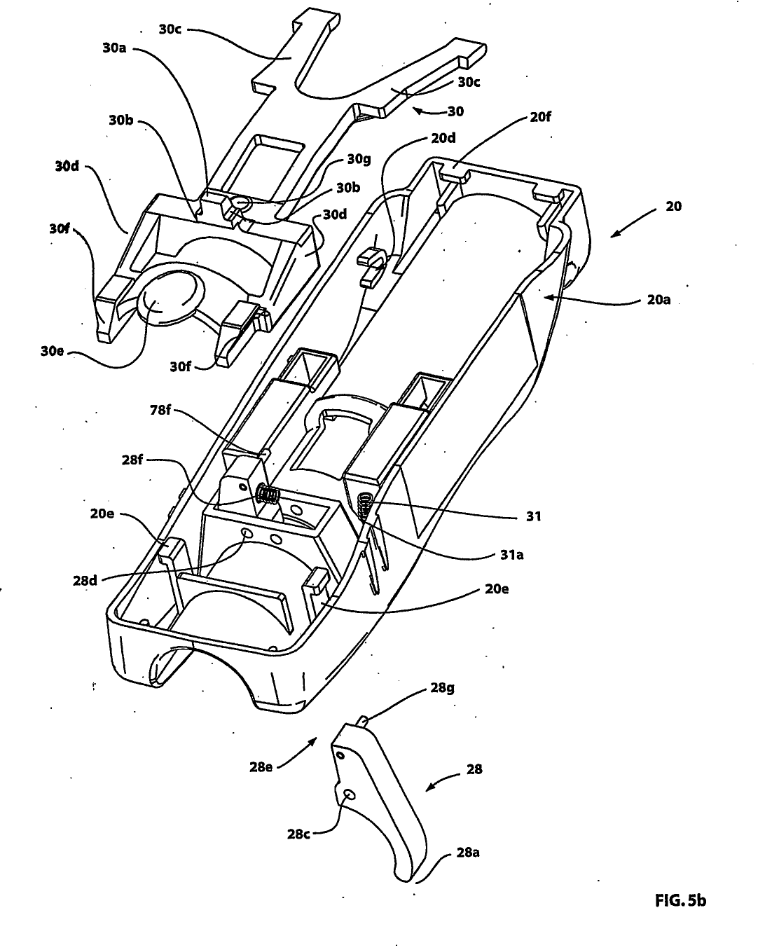

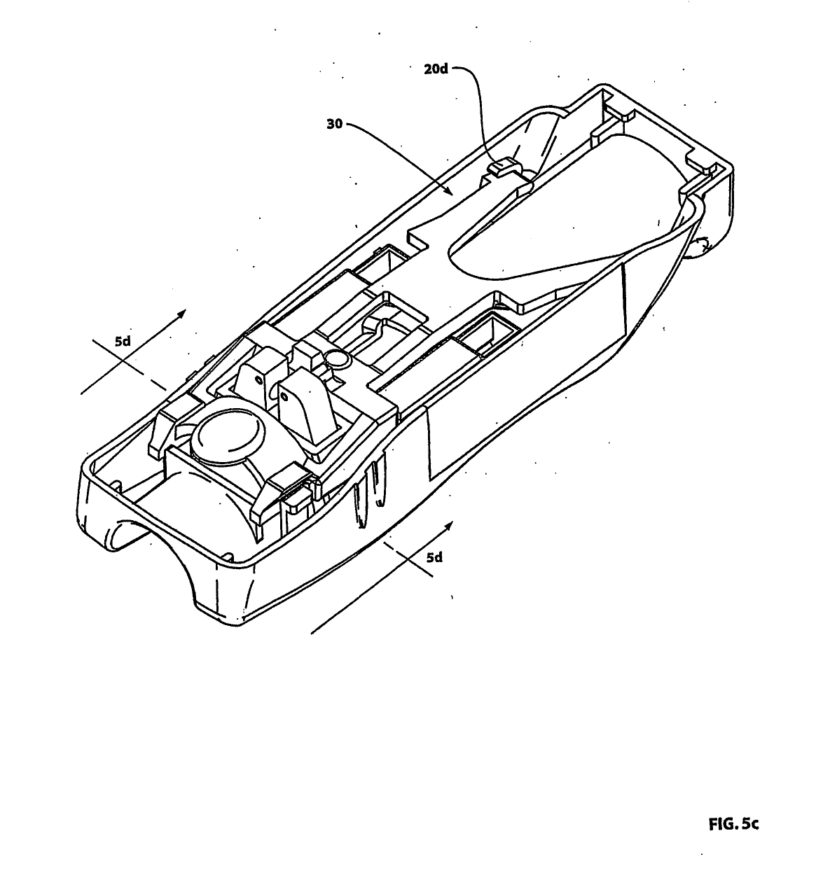

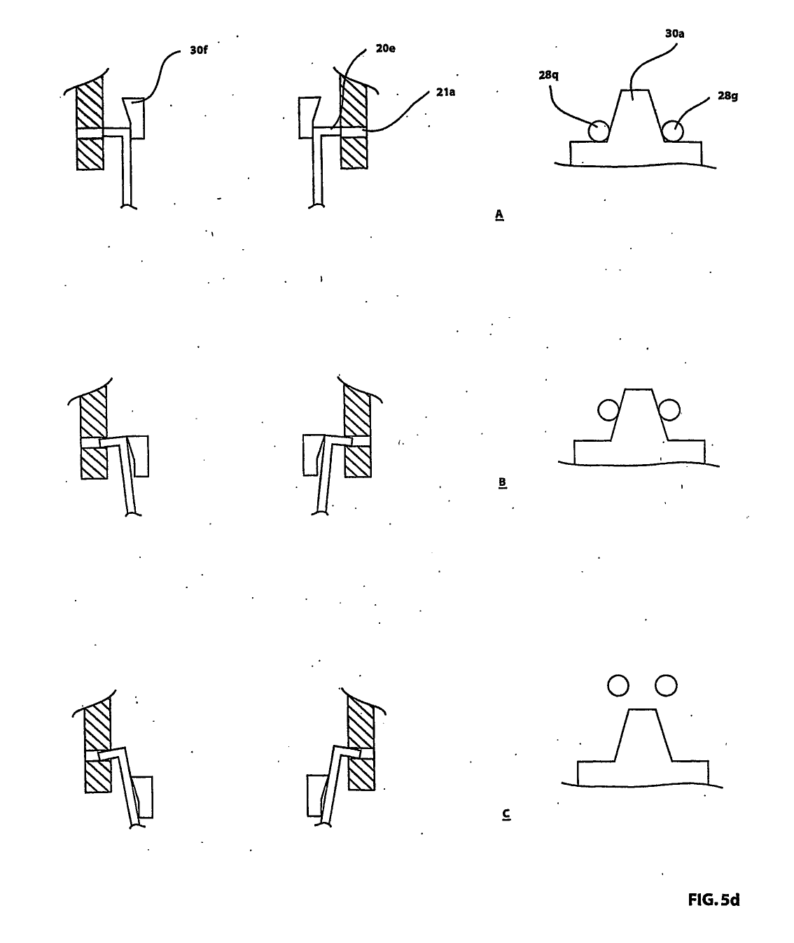

Referring to FIG. 5b, each of the barrier members are pivoted by way of a pivot pin 28c received in passage 28d for mounting each of said barrier members to the body for movement between the release and lock positions.

The barrier members 28 are, in this case, spring biased toward the lock position, by way of spring 28f. This means that even if the syringe carrier is in the release position, the barrier members 28 will retain the syringe in the cavity until needed, at which time the syringe may simply be grasped from the carrier and removed therefrom, against the biasing force of the spring 28f.

Each barrier member has an inner end region 28e opposite the outer free end region 28a and the inner end regions 28e cooperate with a latch means, as will be described, for establishing the release and lock positions of the barrier member 28.

Referring to FIGS. 5a and 5b, the syringe engaging portion 20 includes a body 20a and a trigger structure 30 movably mounted on the body 20a. The control portion 21 further includes a solenoid actuated driver 32 (as shown in FIG. 5a), wherein the trigger structure 30 is movable relative to the body 20 under the action of the actuating driver 32, which engages the trigger structure 30 at an actuating pad shown at 30g, in FIG. 5b.

The trigger structure 30 is pivotally coupled with the body 20a. As is best seen in FIG. 5b, the trigger structure 30 includes a latch portion 30a providing a pair of opposed latch surfaces 30b. The inner end regions 28e of each barrier member 28 includes a locking pin 28g which is either obstructed by the latch portion 30a (thus locking the barrier portions from being transferred to their release positions) or not, depending on the position of the trigger structure 30. The trigger structure 30 includes a first pair of support arms 30c engaging a corresponding pair of pivot locations in the body, one of which is shown at 20d in FIG. 5b. The trigger structure 30 has a pair of second arms 30d supporting a user-activated trigger pad 30e. Thus, the latch portion 30a is centrally located between the first and second arms 30c and 30d.

Referring to FIGS. 5a and 5b, the syringe engaging portion 20 has a pair of locking tabs 20e to engage a corresponding pair of complementary locking tab-receiving cavities 21a on the control portion 21. In addition, a pair of locking tabs 20f match a corresponding set of tab-receiving cavities 21b on the control portion 21.

The second arms 30d have free ends with guide members 30f, each with an inclined surface which is aligned with a corresponding locking tab 20e. Therefore, when the trigger structure 30 moves downwardly, each guide member engages a corresponding locking tab 20e and displaces it toward an engaged position with a corresponding tab-receiving cavity 21a.

The trigger structure 30 is outwardly biased from the syringe engaging portion by way of spring 31 positioned on spike 31a, as shown in FIG. 5b.

FIG. 5d shows the interaction of the locking tabs 20e and the guide members 30f taken on line 5d-5d of FIG. 5c (with some dimensions exaggerated therein for illustration purposes). The trigger structure 30 is thus operable in an outer first position, as shown at A, in which the operative portion of the guide members 30f are disengaged from the locking tabs 20e, meaning that the locking tabs 20e are also disengaged from the cavities 21a. In this position, then, the syringe engaging portion 20 is disconnected from the control portion 21. Meanwhile, the latch member 30a is extending between the locking pins 28g, thereby placing the barrier members 28 in their lock positions.

The trigger structure 30 is then movable to a second position, as shown at B in FIG. 5d, to cause the guide members 30f to displace the locking tabs 20e into the cavities 21a (when the cavities 21a are in the required position in the first place) thereby to connect the syringe engaging portion 20 with the control portion 21, while the latch member 30a remains between the locking pins 28g to maintain the barrier members 28, still, in their respective lock positions.

The trigger structure is moveable to its third position, as shown at C in FIG. 5d, in which the locking tabs remain in the cavities but the latch member 30a is no longer between the locking pins 28f, so that the barrier members may now be moved to their release position against the biasing action of the spring 28f.

Thus, the syringe engaging portion 20 enables the syringe to be held therein by deploying the trigger structure 30 between the first, second or third positions which can be done either manually with a user-actuated pad 30e or by an actuator 32 via an actuating pad 30g. In the latter case, the solenoid actuator 32 has a retracted position, in which its free end is either flush with or beneath a surface 21e of the control portion 21 surrounding it. This position corresponds to the first position of trigger structure 30.

The actuator 32 may then be extended to a middle position corresponding to the trigger structure's second position. Finally, the actuator 32 may then be displaced to a fully extended position wherein the actuating member has displaced the trigger structure 30 to its third position.

The syringe engaging portion 20 is locked in position on the control portion 21 by aligning, first, the locking tabs 20f with the corresponding tab receiving cavities 21b and then by aligning the locking tabs 20e with the corresponding tab-receiving cavities 21a, with the trigger structure in its first position. The movement of the actuator, then, from its retracted position to its middle or fully expended positions causes the locking tabs to engage the cavities which, as mentioned previously, correspond to the trigger structure's second and third positions.

As mentioned earlier, the trigger structure is also conveniently operable between the first, second and third positions when the syringe engaging portion is removed from the control portion, simply by the user depressing the trigger structure at the user-activated pad 30e.

The attachment, if desired, may also be configured so that the syringe-engaging portion 20 must remain with the control portion during the blood treatment process, so that any unauthorized removal of the syringe-engaging portion may, for example, lock the first syringe in the syringe-engaging portion.

Referring to FIGS. 3d, 5a and 5b, the control portion 21 includes first sensing means 34, in the form of a proximity detector, for sensing the presence of the syringe engaging portion 20, in a lock position with the control portion 21. The control portion 21 also includes second sensing means 36 for sensing the presence of at least one type of syringe in the syringe engaging portion 20. The second sensing means 36 is, for example, a proximity detector and the syringe engaging portion 20 has an opening 36a (as shown in FIG. 3d) to align with the proximity detector 36, which makes the proximity detector 36 fully exposed to the syringe receiving cavity 24.

The syringe is then to be equipped with a label having a sector which is either dark or light, either of which registers a different result by the proximity detector 36. Furthermore, the proximity detector is capable of sensing two regions on the label in order to be able to detect either a pre-treatment syringe (referred to by the reference “S1”) carrying untreated blood and a post-treatment syringe (identified by the reference “S2”), by giving each corresponding label a different combination of dark regions and light regions to be detected by the proximity detector 36. The control portion 21 includes data port 37, as shown in FIG. 3c, for exchanging data with a blood treatment unit.

The system is also provided with a wristband 38 (shown in FIG. 1) which contains a barcode that shares a common data component with the first syringe S1, so that each can be linked with a common patient. The barcodes on the wristband and first syringe S1 may also each include an additional data component, respectively identifying each as such. The wristband 38 and the first syringe S1 also include an area or location to receive indicia in printed or written form, in a manual or automated manner, identifying the patient with such information as the patient's name, data of birth and the like in a manner readable to both an operator and a patient, so that a patient, for example, may be located in a busy waiting room.

Referring to FIG. 5, the control portion 21 includes a data transfer unit 40 in the form of a printed circuit board with various chips mounted thereon and powered by batteries 40a. The data transfer unit 40 includes data receiving means 42 in the form of a barcode reader and data storage means 44 in the form of a memory chip or the like for recording and storing data received by the data receiving means. The data receiving means 42 may include a wired or wireless data port. The wireless data port may include, as an alternative, an RF or Infrared signal transmitter or receiver, for example.

The data transfer unit 40 is operable to receive patient identification data representative of a subject patient and thereby to establish a first blood transaction condition, namely by scanning the patient's wristband 38 with the barcode reader 42. The control portion 21 is operable in the first blood transaction condition to transfer the barrier members to the release position to receive the first syringe S1 containing blood from the subject patient and to transfer the barrier members to the lock position to lock the first syringe S1 in the cavity.

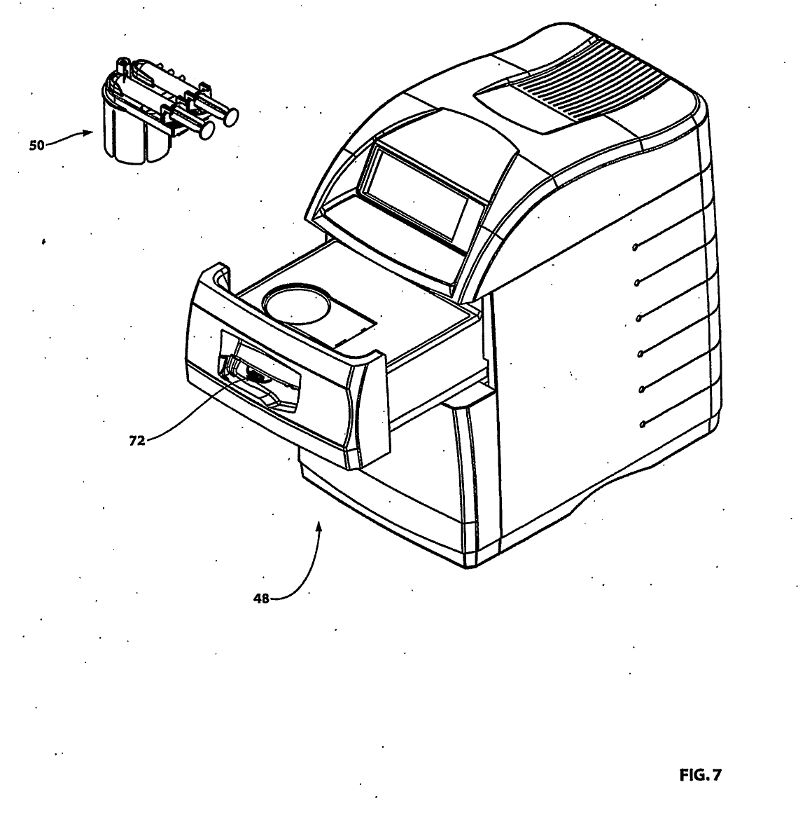

Referring to FIGS. 2 and 7, the blood treatment unit 14 has an access port 48, in the form of a drawer on the front face of the unit, which may be opened to expose an inner blood sample receiving area. A syringe platform 50 is provided to position the first syringe S1 in the inner sample receiving area. Preferably, the syringe platform 50 is disposable and forms part of a blood treatment package as will be described.

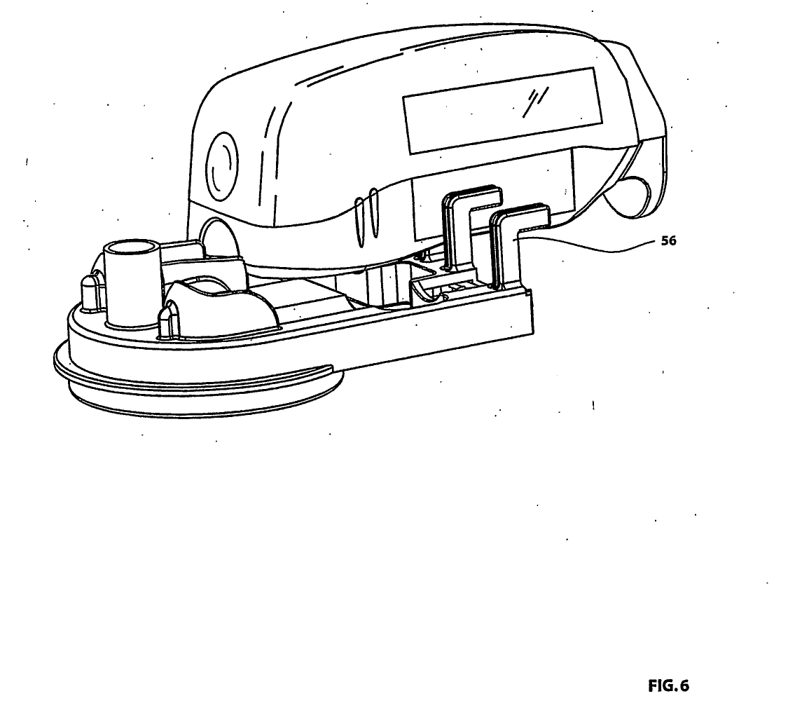

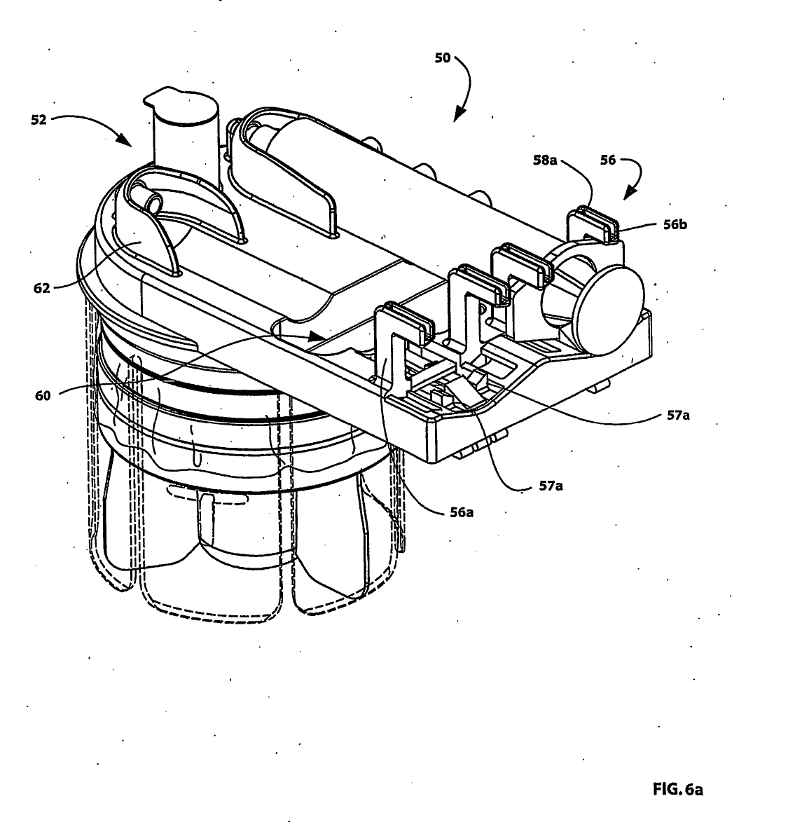

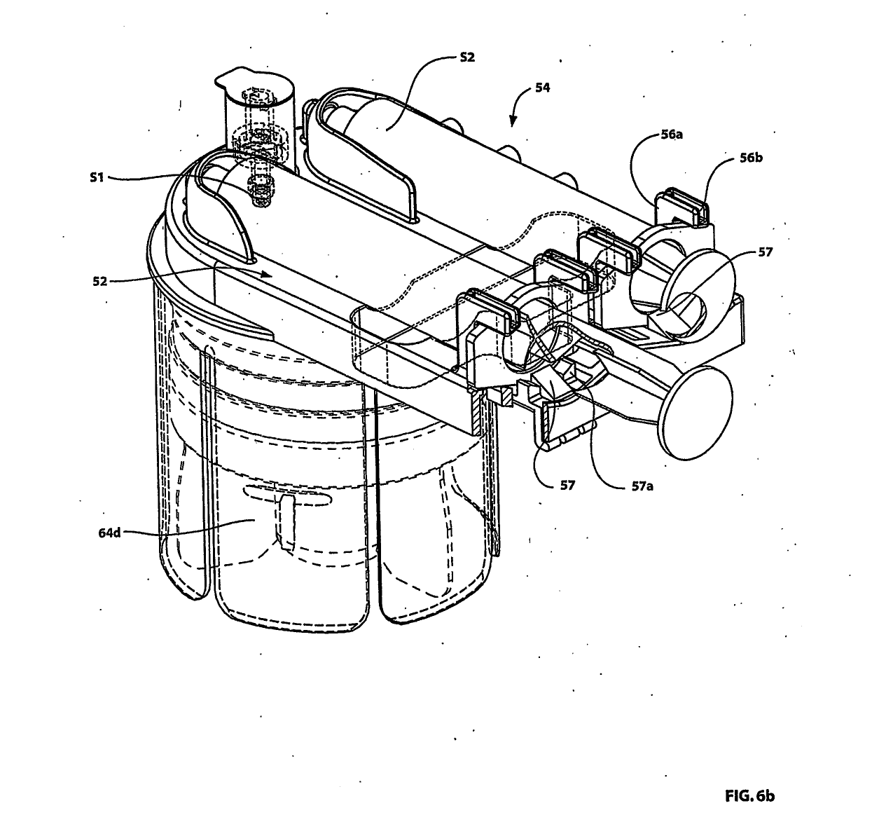

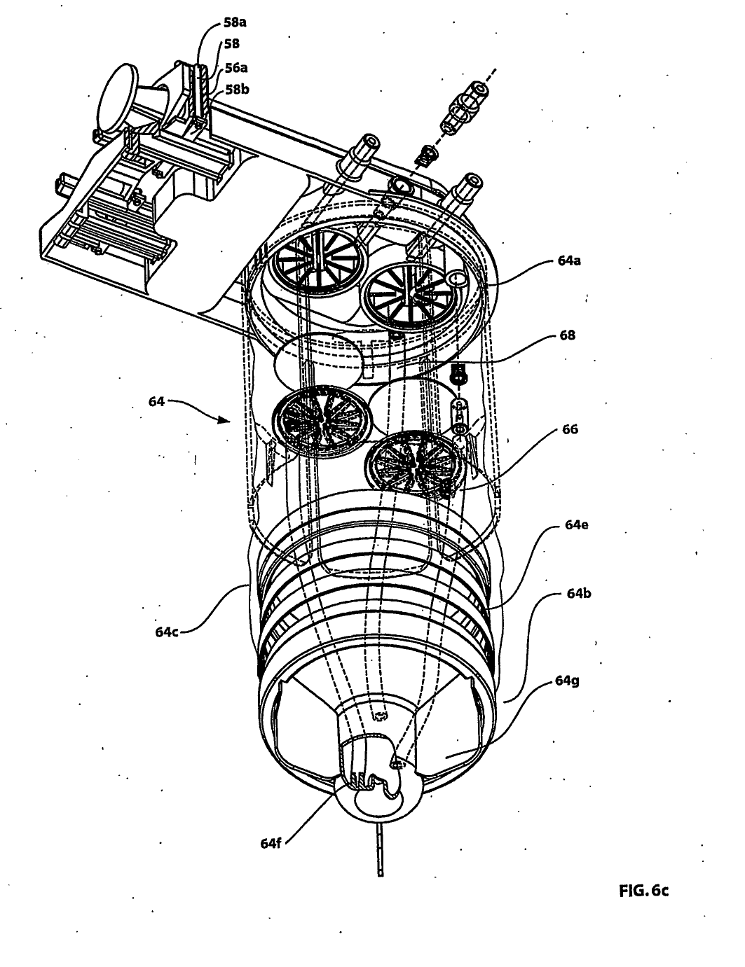

Referring to FIGS. 6 and 6a to 6c, the syringe platform 50 has a first syringe station 52 to receive the first syringe “S1” and a second station 54 to receive a second syringe “S2” as will be described herein below. The syringe platform 50 is further provided with anchor means for anchoring the first and second syringes thereto, in the form of a pair of upstanding anchor members 56, as shown in FIG. 6.

The anchor members 56 are right angled in a manner permitting the syringe flange to fit beside and beneath each A locking tab 57 is positioned in the syringe path of each syringe to lock it in place. Referring to FIGS. 6a to 6c, an actuating member 58 is provided for releasing the locking tabs 57 from the syringe path to allow the syringe to be located in the corresponding syringe station 52, 54. Each anchor member 56 has an upright inner passage 56a and each actuating member 58 includes an actuating post 58a which is slidably mounted in a corresponding inner passage and of sufficient length to emerge from an upper surface of the anchor member as will be described.

The anchor members 56 are also equipped to permit the carrier 12 to establish a physical “syringe transfer link” therewith to facilitate proper transfer of the syringe to the appropriate station. To achieve this, the anchor member 56 has an alignment groove 56b which matches a corresponding ridge 20b (shown in FIG. 3d) in a second locating cavity 24d formed on the syringe engaging portion 20.

Referring once again to FIG. 6c, the actuating member 58 has a cross member 58b which joins the lower ends of the posts 58a and lies adjacent the respective locking tab 57, so that downward displacement of the post causes a corresponding downward movement of the cross member, in turn causing a downward flexing movement of the locking tab 57 to open the syringe path to receive either syringe S1 or S2. The post is displaced by a corresponding ridge 20b on the syringe engaging portion.

The syringe station 52 is also provided with at least one, in this case two, permanent locking tabs 57a which are unresponsive to the actuating member 58, as shown in FIGS. 6a and 6b. These locking tabs are operable to lock the syringe S1 permanently in the platform 50.

Referring to FIG. 6a, each syringe station includes a spill collecting chamber 60 for collecting spilled materials from the corresponding syringe. Each station 50, 52 further includes a pair of syringe fluid transfer terminals 62, each to establish fluid communication with a corresponding one of syringes S1, S2.

Referring to FIGS. 6a to 6c, the syringe platform further includes an expansible treatment chamber 64 which is shown in its collapsed condition in FIG. 6a and in its expanded condition in FIG. 6c. Each syringe fluid transfer terminal is in fluid communication with said treatment chamber to deliver untreated blood thereto, or to withdraw treated blood therefrom. The syringe platform further includes a pair of conduits 66, 68, each joined at one end to a corresponding syringe fluid terminal.

Referring to FIG. 6c, the treatment chamber 64 includes an upper lid portion 64a, a lower base portion 64b, a collapsible jacket portion 64c there between, all of which is nested in a sleeve 64d as shown in FIG. 6b. The collapsible jacket portion 64c also includes at least one, in this case three, positioning rings 64e on the jacket portion to ensure an orderly collapsing of the jacket into the sleeve 64d. The base portion 64b includes a pair of fluid transfer flanges 64f for receiving one end of each of the conduits thereon, each of the fluid transfer flanges establishing fluid communication between an interior region of the treatment chamber and each of said conduits.

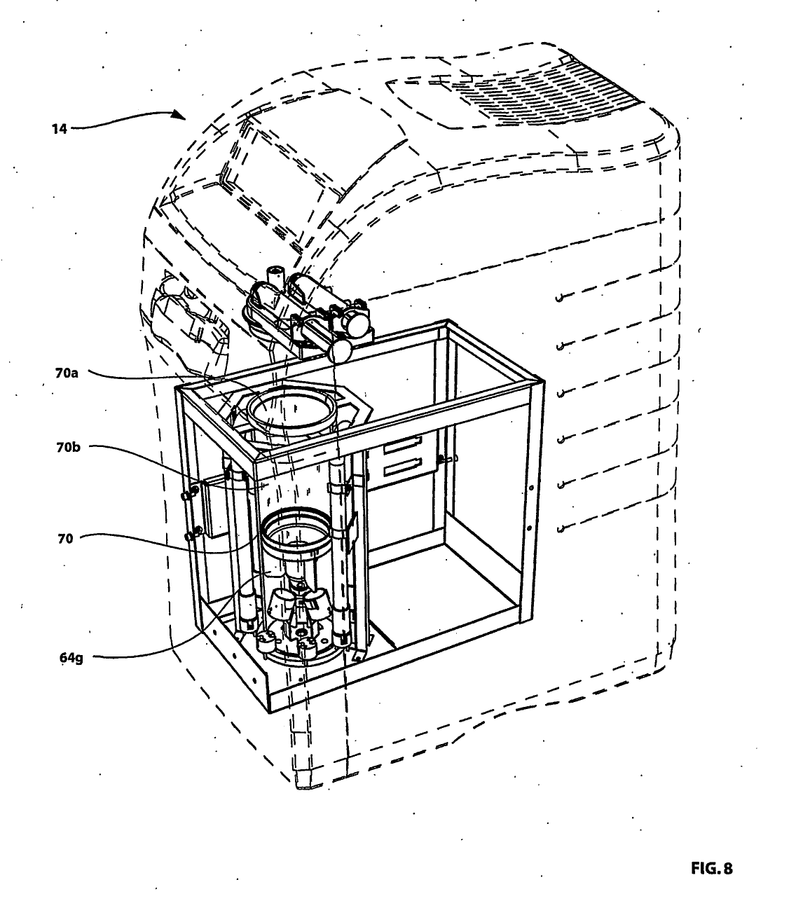

Referring now to FIG. 8, the blood treatment unit 14 has a positioning housing 70 with an inner passage 70a to receive the treatment chamber 64. The positioning housing 70 includes a transparent cylindrical housing portion 70b whose inner cross sectional area is selected to nest the treatment chamber therein. The lower base portion 64b includes a number of positioning vanes 64g extending downwardly therefrom, the vanes dimensioned to align the lower base portion relative to the inner passage,

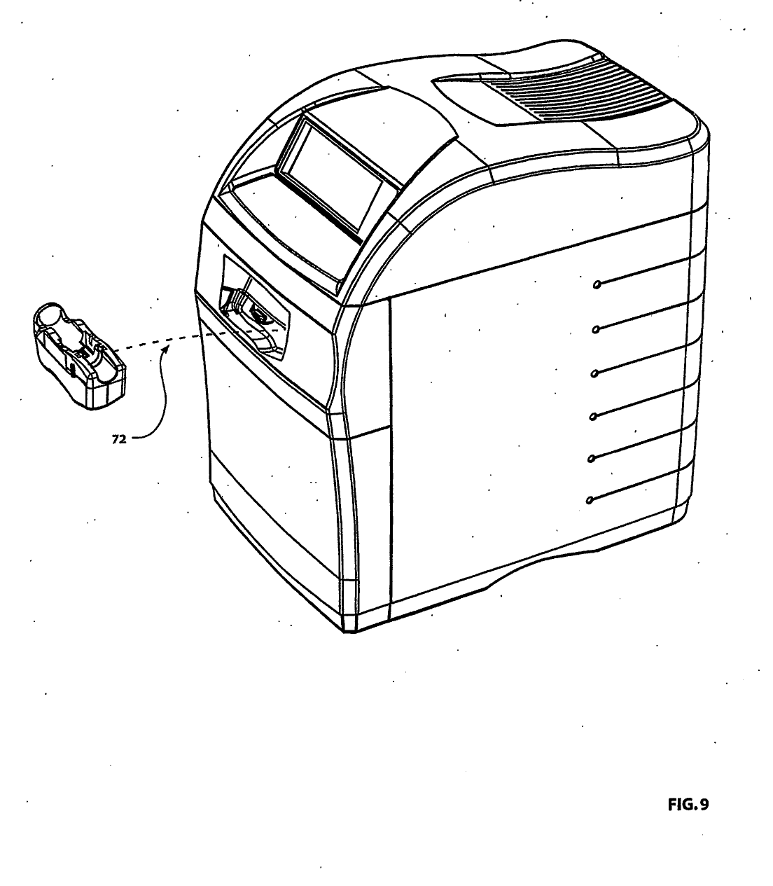

The blood treatment unit 14 includes a syringe carrier docking bay at 72 as shown in FIGS. 7 and 9, which receives the syringe carrier 12 following its release of the first syringe S1. The docking bay 72 includes a data port to be coupled with the complementary data port 37 (shown in FIG. 3c) in the housing of the control portion 21 to establish a data link between the data transfer unit 40 and a control system within the blood treatment unit 14. The data link between the syringe carrier 12 and the treatment unit may, alternatively, be wireless and use for example, the protocols mentioned below.

The data transfer unit 40 (FIG. 5) is operable to transfer the patient identification data to the blood treatment unit 14 and thereby to establish a second blood transaction condition, in which the control portion is operable to transfer the barrier members to the release position to release the first syringe S1 to the first syringe station 52.

The data transfer unit 40 (FIG. 5) is also operable to receive treated blood sample identification data from the blood treatment unit 14, while the syringe carrier 12 is positioned in the docking bay 72 (FIGS. 7 and 9). The data transfer unit 40 is also operable to receive treated blood verification data from a second syringe containing treated blood from the subject patient and positioned at a second syringe station 54 in the platform 50, thereby to establish a third blood transaction condition. In this case, the barcode reader 42 may be used to scan a barcode located on the second syringe S2. The control portion is operable in this third blood transaction condition to transfer the barrier members 28 to the release position to receive the second syringe.

The data transfer unit 40 is operable to receive patient verification data such as by using the barcode reader 42 to scan a barcode on the patient's wristband to establish a fourth blood transaction condition. The control portion is operable in the fourth blood transaction condition to release the syringe engaging portion 20 from the control portion 21, thereby permitting the operator to carry the second syringe conveniently in the syringe engaging portion 20 to the patient. Alternatively, if desired, the control portion may be operable in the fourth blood transfer condition to transfer the barrier members to the release position to release the second syringe.

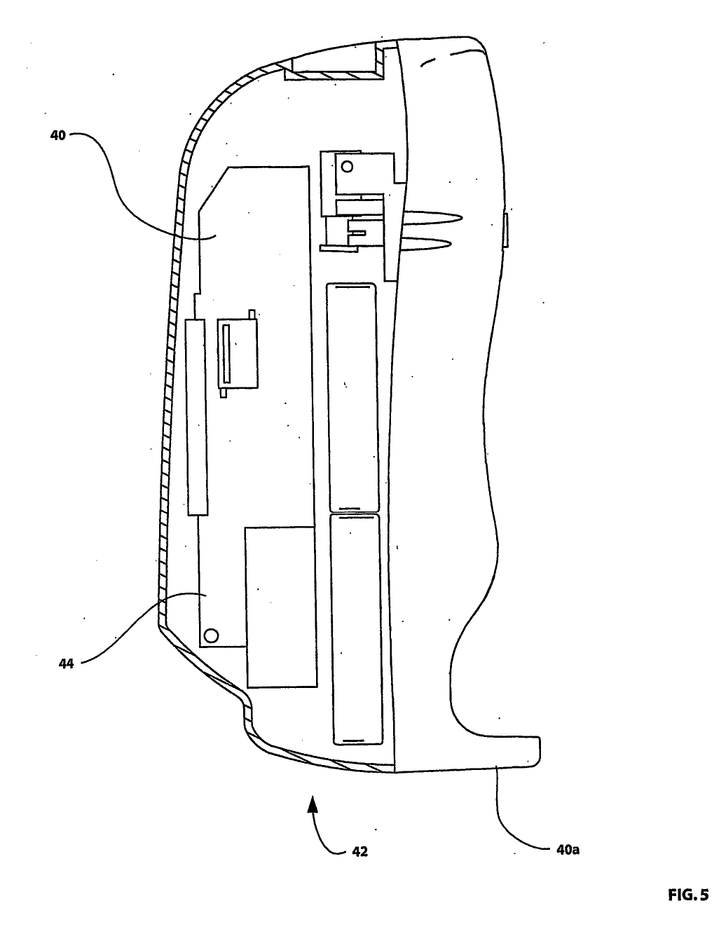

The control portion 21 includes a controller to control the functions of the syringe carrier 12 under the control of a number of preset instructions provided to the control portion 21 by a key pad having one or more buttons, such as that shown at 80 with a single button, located on an external portion of its housing, as shown in FIG. 4. Alternatively, the controller may be responsive to the data being received by the barcode reader which may include specific operational instructions. Alternatively, the data received by the barcode reader may initiate a preset sequence of events where the sequence includes one or more of the blood transaction conditions as described above.

The control portion 21 may include a programmed logic controller or some other form of controller. It may be included in a software program configured to run on a general purpose computer, such as personal computer, or on a more substantial computer mainframe. The control portion 21 may include a computer which as operable to work within a network, for example so that the syringe carrier can be remotely programmed and its collected data uploaded to a central database. The network may thus involve several general purpose computers, for example those sold under the trade names APPLE™ or IBM™, or clones thereof, which are programmed with operating systems known by the trade names WINDOWS™, LINUX or other well known or lesser known equivalents of these. The system may involve pre-programmed software using a number of possible languages or a custom designed version of a programming software sold under the trade name ACCESS™ or similar programming software. The computer network may be a wired local area network, or a wide area network such as the Internet, or a combination of the two, with or without added security, authentication protocols, or under “peer-to-peer” or “client-server” or other networking architectures. The network may also be a wireless network or a combination of wired and wireless networks. The wireless network may operate under frequencies such as those dubbed ‘radio frequency’ or “RF” using protocols such as the 802.11, TCP/IP, BLUE TOOTH and the like, or other well known Internet, wireless, satellite or cell packet protocols. The control function of the control portion 21 may, alternatively, be executed on a single custom built computer which is dedicated to the function of the system alone.

The system may be used in the following manner to control the transfer of blood between a subject patient and the blood treatment unit 14.

A first package is prepared including the patient wristband 38 and the first “blood retrieval” syringe S1. If desired, the first package may also include a disposable or reusable syringe engaging portion 20. A second package may also be prepared including the syringe platform 50 and a second “blood delivery” syringe S2. If desired, both the first and second packages may be prepared in advance with the second syringe S2 locked on the second station 54 of the syringe platform 50, in which the second syringe S2 may only be removed by the syringe carrier 12 during the blood treatment process as will be described.

The first “blood retrieval” syringe S1 contains a barcode similar to the wristband 38 but different enough from the wristband to be acknowledged as such. In other words, both the wristband 38 and the first syringe S1 are provided with a common or generic data component, while the wristband contains a unique data component identifying it as a wristband and the first syringe contains a unique data component identifying it as a first syringe. The second “blood delivery” syringe S2 is fitted with a separate unrelated barcode. Further, a human readable name matching system is also used to follow a blood sample through the steps of the treatment. In other words, the wristband 38 and first syringe S1 are each provided with a label where the patient's name can be added as indicia recognizable to the operator conducting the treatment. The syringe carrier 12 is provided to take control of the delivery of the blood samples to the treatment device and then return to the patient.

The procedure begins with blood being drawn into the first syringe S1, following a previous treatment with sodium citrate, and then the first syringe S1 is capped. The syringe engaging portion 20 is attached to the control portion 21 of the syringe carrier 12, before or after which the control portion 21 is activated and the barcode on the wristband is scanned with the barcode reader. Since the wristband 38 and the first syringe S1 have a common data component, it may be sufficient simply to scan the wristband 38, though the first syringe S1 may also be scanned, if required, for further verification.

As a result, the syringe carrier 12 now has, within its memory, the common data component read by the barcode reader 42. The syringe carrier 12 then unlocks the barrier members and the filled syringe is then positioned in the cavity by spreading apart the now unlocked inwardly syringe biased barrier members 28, with the first and second end flanges in their corresponding first and second formations. The syringe carrier 12 is then activated and the barrier members are actuated to their lock positions to lock the first syringe S1 in place, thus signifying that the first blood transaction condition has been met. This may, for example, occur as a result of a sensor located within the syringe receiving cavity, that senses the presence of syringe S1 and possibly an additional scan of the bar code on syringe S1 for verification purposes. The syringe carrier 12 is then positioned adjacent the platform 50 so that the first syringe S1 can be delivered to the first station 52 thereon and held by the anchor tabs 56 using a sliding action.

Now free of the first syringe S1, the syringe carrier 12 is installed in the syringe carrier docking bay 72 and then transfers patient identification data thereto over the data link which is established between the syringe carrier 12 and the blood treatment unit 14. The blood treatment unit 14 then proceeds to carry out a designated blood treatment on the blood sample, such as that described in as that disclosed in PCT application serial number PCT/CA00/01078 filed Sep. 15, 2000 entitled APPARATUS AND PROCESS FOR CONDITIONING MAMMALIAN BLOOD (the entire contents of which are incorporated herein by reference).

Thereafter, the blood treatment unit 14 delivers the treated blood to the second syringe S2 already positioned in the second station 54 of the platform 50. The second syringe S2 has its own barcode containing a unique data component, which is unrelated to the common data component in the barcode of the first syringe S1 and the wristband 38. The treatment unit then reads the barcode on the second syringe S2 and transfers the treated blood sample identification data contained in the barcode to the syringe carrier 12 through the data transfer port of the docking bay 72. Then, the syringe carrier is positioned so that the barcode reader can scan the treated sample identification on the second syringe S2 to conform a match, at which point the barrier members 28 are released and the second syringe S2 is transferred from the second station 54 in the platform 50 to the cavity and held therein by the barriers 28 in the lock position.

The syringe carrier 12 is then returned to the patient where the barcode reader is scanned over the wristband 38 to confirm a match between the treated blood sample and the subject patient. With the match established, the barrier members 28 may be transferred to their release position and the second syringe removed so that the treated blood may be delivered to the patient, to complete the process. At this point, the syringe engaging portion 20 may be released from the control portion 21 and the syringe engaging portion 20 discarded, along with the S1, S2 syringes and the platform 50. This ensures that all working parts of the system which are intimately associated with a blood sample can be disposed of while retaining other components of the system for re-use.

Thus, the data contained in the barcode and written or printed on the labels of the wristband 38 and the syringes are used to match and track the patient and the blood sample. The wristband contains the subject patient's name and Barcode ID. The syringe carrier 12 obtains and contains the barcode matching information as well as the written or printed patient name information thereon and the operator uses the barcode reader as a secondary matching device.

In addition, the syringe carrier 12 obtains data relating to the blood treatment which may merely record the time at which the blood treatment occurred. If desired, the syringe carrier 12 may also be configured to lock the syringe carrier, if a subsequent step in the blood treatment procedure has not been executed. For example, the syringe carrier may have a lock function triggered by the lack of a status signal received at each stage in the process. In this case, the syringe carrier may also be configured to release the lock after a predetermined access sequence is entered in the carrier, for example via the docking bay 72.

Thus, in addition to the control of untreated and treated blood, samples, the syringe carrier may also accumulate audit trail data which may be uploaded to the blood treatment unit or some other intermediate device following a blood treatment procedure, wherein the audit trail data may be used to monitor the blood treatment to be sure that it was appropriate for the patient's particular condition. The audit trail data may, for instance, be analyzed over the course of a patients short term or long term treatment program, as needed.

The RF ID-Assisted Tracking with Name Label

In this example, the subject patient is fitted with a disposable RF ID scanner on his wrist, or elsewhere inside or outside his body, either attached with or spaced therefrom and the first and second syringes are equipped with RF ID chips within them, for example as shown schematically at 100, 102, 104 in FIG. 1. If desired, written or printed name labels may be affixed on the wristband as well as the first and second syringes.

The treatment may proceed as before, except that the syringe carrier does not function to lock the first or second syringes in place. Rather, the verification function occurs between the wristband, the first syringe, the treatment unit and the second syringe.

The RF ID treatment procedure is proposed as follows. First, the wristband 38 and the first blood retrieval syringe S1 are arranged so that each emits a common RF signal. The patient name and date of birth are written on the wristband as well as on the first and second syringes. A blood sample is then drawn from the patient using the first syringe and the wristband is attached to the wrist of the subject patient.

The syringe is delivered to the first station of the platform which is now in position in the treatment unit. At this point, the second delivery syringe is already installed in the second station of the platform. The treatment occurs where the treated blood sample is injected into the second delivery syringe. As shown by the dashed arrow at 106, the treatment unit reads the RF ID on the first syringe and writes that ID onto the RF Tag of the second delivery syringe (as shown by the dashed arrow at 108), as well as other data as described above such as a time stamp indicative of when the treatment occurred or other steps in the process as the case may be. This process of ID writing may only be done once, with current RF ID chips, though other devices may be available to make the writing process repeatable

The treatment is completed and the treatment unit opens to deliver a platform with an attached and empty first syringe and the attached second syringe containing the now treated blood sample. The second syringe is removed from the platform in the access port and is then transported to the patient. The patient is identified by name using the patient label on the second syringe.

The operator confirms the subject patient's identification by attempting to match RP ID data in the syringe with that contained in the patient's wristband by placing them in close proximity to one another. The wristband RF ID reader will emit a signal to confirm the match. The signal may be a sound or light emission, such as from a signal generator, an LED or the like.

The treated blood is then delivered to the patient. The wristband is removed from the patient and is taken to the treatment unit to close the audit trail and confirm that the treatment was completed.

The RF ID Assisted Tracking without Name Label

In this case, the RF ID based treatment procedure is as follows. First, a package is prepared containing a wristband, syringe carrier and first blood retrieval syringe. The wristband and the blood retrieval syringe have the same factory-installed and matching RF ID's. The patient's name and date of birth are written on the wristband and on a label provided on the syringe carrier.

The first syringe is used to draw a blood sample from the subject patient. The wristband is attached to the subject patient

The syringe carrier is used, as before, to deliver the blood to the first station on the platform which is carrying the second syringe in the second station and the platform is itself located in the blood sample receiving area in the access port 48. The treatment unit is then activated to conduct a designated treatment on the blood sample. Thereafter, the treated blood is delivered to the second syringe.

The treatment unit reads the RF ID on the first syringe and writes that ID onto the RF ID of the second syringe. With the treatment completed, the treatment unit opens to deliver the platform with the two syringes. The operator manipulates the syringe carrier to transfer the barriers to the release position and then fixes the second syringe in the cavity, then transfers the barrier members to the lock position, then removes the second syringe from the disposable. The syringe carrier is then taken to the patient, as identified by the information contained on the syringe carrier label.

The operator confirms the patient's identification by attempting to match the second syringe's RF ID to the wristband RF ID by placing them in close proximity to one another. The wristband RF ID reader will emit a signal, such as a beep and/or a light pulse, a signal over a wired or wireless data network, or the like to confirm a match, at which point the syringe carrier may be activated to transfer the barrier members to the release position so that the second syringe may be removed and the treated blood sample administered to the patient.

The wristband is removed from the patient and is taken to the treatment unit to close the audit trail and confirm that the treatment was completed.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

While the syringe carrier 12 uses barrier members 28 which physically grasp or block the first and second syringes in the cavity, other forms of barriers may be employed, such as those utilizing other physical barrier arrangements or non-physical barrier arrangements. For example, the barrier may be provided as an electromagnet to clamp the syringe in the cavity by way of a magnet coupling with a ferromagnetic band on the syringe body.

The system is applicable to other medical dispensers such as IV bottles, powder and/or atomized fluid and/or gas inhalant dispenser, implant delivery dispenser, ventilator, syringe pump, intubation tube, or a gastrointestinal feeding tube or a plurality and/or a combination thereof. In this case, the dispenser-engaging portion may have a housing with an internal cavity in which the dispenser is entirely or partially concealed and locked therein, or an external cavity or some other formation which grips the dispenser in such a manner that the dispenser is rendered inoperable and/or, inaccessible under controlled treatment conditions.

1-70. (canceled)

71. A device for controlling the collection and delivery of materials to a patient, comprising:
a dispenser carrier,
a dispenser-engaging portion on said carrier, the dispenser-engaging portion being movable between a release position in which it can receive a materials dispenser when the dispenser is in a materials-containing configuration, and a lock position in which a dispenser can be locked thereto,
and access control means operable to control movement of the dispenser-engaging portion between its release position and its lock position according to a materials transaction condition.

72. A device as defined in claim 71 wherein the dispenser-engaging portion includes a side wall and a first cavity to receive the dispenser, the first cavity being located in the side wall and accessible therethrough.

73. A device as defined in claim 72, wherein the dispenser is a syringe and the dispenser-engaging portion is a syringe-engaging portion, and wherein the syringe engages with the syringe-engaging portion on the carrier, the syringe having a body having a first end flange on one end thereof and a plunger slidably engaged with the body, the plunger having a second end flange on a remote end thereof, the cavity having a first formation to receive the first end flange.

74. A device as defined in claim 73, wherein the access control means further comprises at least one barrier portion to extend at least partially across the first cavity in the lock position.

75. A device as defined in claim 74 wherein the access control means further comprises a pair of barrier members with opposing outer free end regions, the barrier members being movable between a release position wherein the free ends are separated to permit the syringe to pass therebetween and a lock position wherein the outer free ends are positioned sufficiently close to one another to prevent removal of the syringe from the first cavity.

76. A device as defined in claim 75 further comprising a control portion, the syringe-engaging portion being removably attached to the control portion.

77. A device as defined in claim 76 further comprising pivot means for pivoting each barrier member between the release and lock positions.

78. A device as defined in claim 77, further comprising biasing means for biasing the barrier members toward their respective lock positions.

79. A device as defined in claim 78 wherein each barrier member has an inner end region opposite the outer free end regions, and further comprising a latch member for latching the barrier members in their lock positions.

80. A device as defined in claim 79, wherein the syringe-engaging portion further comprises a body and a trigger structure movably mounted on the body, and the control portion further comprising an actuating driver, the trigger structure being movable relative to the body under the action of an actuating driver.

81. A device as defined in claim 80 wherein the trigger structure is pivotally coupled to the body, the latch member being positioned to obstruct the path of the inner end regions of the barrier members in their lock positions.

82. A device as defined in claim 81 wherein the trigger structure includes a latch portion, the inner end regions of each barrier member each including a locking pin which is obstructed by the latch member in the lock position.

83. A device as defined in claim 82 wherein the trigger structure includes a first pair of support arms engaging a corresponding pair of pivot locations in the body and at least one second arm including a user-activated trigger pad.

84. A device as defined in claim 83 including a cam portion centrally located between the second arm on one side thereof and the two first arms on another side thereof.

85. A device as defined in claim 84 wherein the trigger structure is operable in a first position to disconnect the syringe-engaging portion from the control portion, a second position to connect the syringe-engaging portion with the control portion with the barrier members in their respective lock positions and a third position to connect the syringe-engaging portion with the control portion with the barrier members in their respective release positions.

86. A device as defined in claim 85 wherein the syringe-engaging portion has at least one locking formation to engage at least one first complementary locking formation on the control portion, the syringe-engaging portion further comprising at least one second locking formation to engage at least one second complementary locking formation on the control portion, wherein the second locking formation is movable to an engaged condition with the second complementary locking formation when the trigger structure is in the second or third positions.

87. A device as defined in claim 86, further comprising a pair of opposed second locking members, the trigger structure further including a pair of abutment portions each arranged to engage a corresponding second locking member.

88. A device as defined in claim 76 wherein the control portion includes a data transfer unit, the data transfer unit being operable to transmit and receive patient identification data representative of a subject patient and thereby to establish a first material transaction condition, the control portion being operable in the first material transaction condition to activate the actuating driver to establish the release position for the barrier members to receive a first syringe containing material from the subject patient and to establish the lock position for barrier members to lock the first syringe in the first cavity.

89. A device as defined in claim 88 wherein the data transfer unit includes data transmitting and receiving means and data storage means for recording data received by the data receiving means.

90. A device as defined in claim 89 wherein the data receiving means includes a wireless data port with a barcode reader, an RF signal receiver or an infrared transmitter receiver.

91. A device as defined in claim 90 for controlling the collection and delivery of blood to and from a patient, and the data transfer unit is operable to transfer the patient identification data to a blood treatment unit and thereby to establish a second blood transaction condition, the control portion being operable in the second blood transaction condition to activate the actuating driver to establish the release position for the barrier members to release the first syringe to a first syringe station in the blood treatment unit.

92. A device as defined in claim 91 wherein the data transfer unit is operable to receive treated blood identification data from the blood treatment unit, the data transfer unit also being operable to receive treated blood verification data from a second syringe containing treated blood from the subject patient and positioned at a second syringe station in the blood treatment unit, thereby to establish a third blood transaction condition, the control portion being operable in the third blood transaction condition to activate the actuating driver to establish the release position for the barrier members to receive the second syringe.

93. A device as defined in claim 92 wherein the data transfer unit is operable to receive patient verification data to establish a fourth blood transaction condition, the control portion being operable in the fourth blood transaction condition to activate the actuating driver to establish the release position for the barrier members to release the second syringe.

94. A device as defined in claim 93 wherein the control portion includes first sensing means for sensing the presence of the syringe-engaging portion.

95. A device as defined in claim 94 wherein the control portion includes second sensing means for sensing the presence of at least one type of syringe in the syringe-engaging portion.

96. A device as defined in claim 95 wherein the second sensing means includes a proximity detector, the syringe-engaging portion having an opening to align with the proximity detector.

97. A system for blood processing, comprising:
a first syringe to receive a blood sample from a subject patient;
a patient identifier attachable to the subject patient;
blood treatment unit;
a syringe carrier for transferring the first syringe containing the blood sample to the blood treatment unit, the syringe carrier being operable in a release position to receive the first syringe when the first syringe is in a blood-containing configuration, the syringe carrier being operable in a lock position for locking the first syringe therewith;
access control means for controlling the release and lock positions to control access to the first syringe according to a blood sample transfer condition;
a second syringe to receive the blood sample after treatment in the blood treatment unit to form a treated blood sample;
the syringe carrier being operable in the release position to receive the second syringe when the second syringe is in a blood-containing configuration, the syringe carrier being operable in the lock position for locking the second syringe therewith;
said access control means being operable to control the release and lock positions to control access to the second syringe according to a treated blood transfer condition.

98. A system as defined in claim 97 wherein the patient identifier includes a patient wristband.

99. A system as defined in claim 97, wherein the syringe carrier includes a syringe-engaging portion with a side wall and a first cavity therein to receive the syringe.

100. A system as defined in claim 99 wherein the first syringe has a body having a first end flange on one end thereof and a plunger slidably engaged with the body, the plunger having a second end flange on a remote end thereof, the first cavity having a first formation to receive the first end flange.

101. A system as defined in claim 100, wherein the access control means further comprises at least one barrier portion to extend at least partially across the first cavity in the lock position.

102. A system as defined in claim 101 wherein the access control means further comprises a pair of barrier members with opposing free end regions, the barrier members being movable between a release position wherein the free ends are separated to permit the first or second syringes to pass therebetween and a lock position wherein the free ends are positioned sufficiently close to one another to prevent removal of the syringe from the cavity.

103. A system as defined in claim 102, further comprising actuating means for actuating the barrier members between the release and lock positions.

104. A system as defined in claim 103, wherein the syringe carrier further comprises a control portion, the syringe-engaging portion being removably attached to the control portion.

105. A system as defined in claim 104 wherein the control portion includes a data transfer unit, the data transfer unit being operable to transmit and receive patient identification data representative of a subject patient and thereby to establish an untreated blood sample transfer condition, the control portion being operable in the untreated blood sample transfer condition to transfer the barrier members to the release position to receive the first syringe containing blood from the subject patient and to transfer the barrier members to the lock position to lock the first syringe therein.

106. A system as defined in claim 105 wherein the data transfer unit includes data receiving means and data storage means for recording data received by the data receiving means.

107. A system as defined in claim 106 wherein the data receiving means is a wireless data port including a barcode reader or an RF signal receiver.

108. A system as defined in claim 105 wherein the data transfer unit is operable to receive treated blood identification data from the blood treatment unit, the data transfer unit also being operable to receive treated blood verification data from the second syringe containing treated blood from the subject patient and positioned at a second syringe station in the blood treatment unit, thereby to establish a treated blood transfer condition, the control portion being operable in the treated blood transfer condition to transfer the barrier members to the release position to receive the second syringe and to transfer the barrier members to the lock position to lock the first syringe therein.

109. A system as defined in claim 107, wherein the treatment unit includes a housing, further comprising a syringe platform removably mounted in the housing, the platform further comprising a first syringe station to receive the first syringe and a second syringe station to receive the second syringe.

110. A system as defined in claim 109 wherein the syringe platform further comprises an anchor means for anchoring the first and second syringes at the first and second syringe stations respectively.

111. A system as defined in claim 110 wherein each anchor means includes at least one upstanding anchor tab which engages the first end flange on the first syringe.

112. A system as defined in claim 111, further comprising an actuating member for displacing the tabs for locating the corresponding syringe in the corresponding syringe station.

113. A system as defined in claim 112 wherein the actuating member includes at least one release pin which is oriented to make contact with the syringe-engaging portion for displacing the release pins when the syringe-engaging portion is in a syringe delivering orientation adjacent the corresponding syringe station.

114. A system as defined in claim 113, further comprising a pair of actuating pins for each syringe station, a pair of alignment flanges on oppose sides of each syringe station, each of said alignment flanges including a longitudinal passage locating one of said release pins.

115. A system as defined in claim 114 wherein each alignment flange includes an upstanding post and a groove to receive a corresponding ridge formed on the syringe-engaging portion.

116. A system as defined in claim 115 wherein the ridge is located in a second locating cavity formed on the syringe-engaging portion.

117. A system as defined in claim 113, further comprising at least one permanent locking flange which is unresponsive to the release pins.

118. A system as defined in claim 117, wherein each syringe station includes an exposed spill collecting chamber for collecting spilled materials from the corresponding syringe.

119. A system as defined in claim 117 wherein the syringe platform further includes a pair of syringe fluid transfer terminals, each to establish fluid communication with a corresponding one of said first and second syringes.

120. A system as defined in claim 109 wherein the syringe platform further comprises a treatment chamber, each syringe fluid transfer terminal being in fluid communication with said treatment chamber.

121. A system as defined in claim 120 wherein the treatment chamber is expansible.

122. A system as defined in claim 121, wherein the syringe platform further includes a pair of conduits, each joined at one end to a corresponding syringe fluid terminal.

123. A system as defined in claim 122 wherein the treatment chamber includes an upper lid portion, a lower base portion and a collapsible portion there between.

124. A system as defined in claim 123 wherein the collapsible portion includes a sleeve and at least one positioning ring between the upper lid portion and the lower base portion.

125. A system as defined in claim 124 wherein the base portion includes a pair of fluid transfer flanges for receiving one end of each of said conduits thereon, each of said fluid transfer flanges establishing fluid communication between an interior region of the treatment chamber and each of said conduits.

126. A system as defined in claim 125, further comprising a positioning housing with an inner passage to receive the treatment chamber, the housing including a transparent cylindrical housing portion whose inner cross sectional area is selected to nest the treatment chamber therein.

127. A system as defined in claim 123 wherein the lower base portion includes a number of positioning vanes extending downwardly therefrom, the vanes dimensioned to align the lower base portion relative to the inner passage.

128. A method of controlling the transfer of blood between a subject patient and a blood treatment unit, comprising the steps of:
providing a first syringe containing a sample of untreated blood from a subject patient;
providing a syringe carrier which is operable in a release position to receive the first syringe; the syringe carrier being operable in a lock position for locking the first syringe therewith, the carrier having an access controller for controlling the release and lock positions according to a blood transaction condition, the access controller including a data transfer unit which is operable to receive patient identification data representative of a subject patient;
in a first blood transaction step, delivering patient identification data representative of a subject patient to the data transfer unit, thereby to place the syringe carrier in a release position to receive the first syringe and thereafter to place the syringe carrier in a lock position to lock the first syringe therein;
in a second blood transaction condition, transferring the patient identification data to a blood treatment unit, thereby to place the syringe carrier in the release position to release the first syringe to a first syringe station in the blood treatment unit;
in a third blood transaction step, delivering treated blood identification data from the blood treatment unit to the syringe carrier, and delivering treated blood verification data from a second syringe containing treated blood from the subject patient and which is positioned at a second syringe station in the blood treatment unit, and placing the syringe carrier in the release position to receive the second syringe;
in a fourth blood transaction step, delivering patient verification data to the syringe carrier and placing the syringe carrier in the release position to release the second syringe.

129. A method of controlling the transfer of blood between a subject patient and a blood treatment unit, comprising the steps of:
providing a first syringe to receive a sample of untreated blood from a subject patient;
providing the subject patient with a patient RF signal processor;
providing a second syringe to receive the sample following treatment;
providing each of the first syringe and the second syringe with an RF signal processor;
arranging the RF signal processors on the first syringe and with the patient to issue a signal containing common or related identity data;
delivering the first syringe to the blood treatment unit for performing a treatment step to form a treated blood sample;
reading the identity data from the first syringe and writing the identity data to the second syringe;
collecting the treated blood sample from the treatment unit in the second syringe;
bringing the second syringe within range of the patient RF signal processor to confirm a match therebetween; and thereafter
delivering the treated blood sample to the patient.
